(19) United States
(12) Patent Application Publication
(10) Pub. No.: US 2004/0015085 A1
(43) Pub. Date: Jan. 22, 2004

(54) Apparatus and method for detecting luminescence from biological systems in response to magnetic fields

(76) Inventors: Kwang-Sup Soh, Gyeonggi-do (KR); Byung-Cheon Lee, Gyeonggi-do (KR); Hyeon-Min Johng, Seoul (KR); Chunho Choi, Seoul (KR); Jung-Ho Lee, Seoul (KR); Jae-Kwan Lim, Seoul (KR); Joon-Mo Yang, Chungcheongbuk-do (KR); Ku-Youn Baik, Gyeonggi-do (KR); Tae-Jeong Nam, Gyeonggi-do (KR)

Correspondence Address: NIXON & VANDERHYE, PC, 1100 N GLEBE ROAD, 8TH FLOOR, ARLINGTON, VA, 22201-4714, US

(21) Appl. No.: 10/619,598

(22) Filed: Jul. 16, 2003

(30) Foreign Application Priority Data
Jul. 19, 2002 (KR) 2002-42429

(51) Int. Cl. A61B 6/00

(52) U.S. Cl. 600/473; 250/458.1

(57) ABSTRACT

This invention of an apparatus and a method for detecting luminescence from biological systems in response to magnetic fields measures the amount of light emitted when impressing a magnetic field on a living thing, a tissue or cells separated from a living thing to evaluate the effect of magnetic fields on biological systems. The apparatus of this invention consists of a dark box which shields a biological sample from external light, a magnetic field generator which is arranged adjacent to the biological sample to impress a magnetic field on it, and a photodetector which detects the light emitted from the biological sample exposed to the magnetic field from the said magnetic generator.

### BACKGROUND OF THE INVENTION

The present invention relates generally to an apparatus and a method for measurement of the effect of magnetic field on biological systems, and more particularly to an apparatus and a method for measuring the amount of light emitted from a biological system in response to impressing a magnetic field on it.

Recently there has been much discussion about the probability of negative effects of electromagnetic fields on biological systems including the human body. But this has not yet been scientifically proven. Many electric appliances such as microwave ovens and cellular phones generate electromagnetic waves, and concerns about harmfulness of electromagnetic waves are increasing particularly with the rapid increase of cellular phone usage.

Amongst electromagnetic waves, magnetic waves cannot be easily shielded, while electric waves can be shielded with relative ease. It is therefore necessary to scientifically examine the effects of magnetic fields on biological systems including the human body.

Since 1990 medical treatments for relieving Parkinson's disease using magnetic field have been developed. Research into remedies for mental diseases such as hypochondria and epilepsy using magnetic fields has also increased. Now, it is necessary to analyze the effects of magnetic fields on the human body and other living things to confirm the therapeutic effects of magnetic fields.

With the notion that biological systems emit extremely small amount of light in natural circumstances, we have invented an apparatus and a method for detecting luminescence from biological systems in response to magnetic fields.

### SUMMARY OF THE INVENTION

It is an object of the present invention to provide an apparatus and a method for detecting luminescence from biological systems in response to impressing a magnetic field on them to examine the effect of the magnetic field.

It is another object of the present invention to provide an apparatus and a method for detecting luminescence from living things as well as tissues and cells separated from living things in response to magnetic fields.

The above and other objects are attained, according to the present invention by an apparatus for detecting luminescence from biological systems in response to magnetic fields comprising a magnetic field generator which is arranged adjacent to a biological sample and generates a magnetic field to be impressed on the said biological sample, a photodetecting device which detects the light from the said biological sample, and a dark box which shields the said biological sample from light outside the dark box. The said biological sample may be tissues or cells separated from a living thing or a living thing itself.

The present invention of the method for detecting luminescence from biological systems in response to magnetic fields comprises the steps of preparing a biological sample, shading the biological sample, impressing a magnetic field on the biological sample, and detecting luminescence from the biological sample.

### BRIEF DESCRIPTION OF THE DRAWINGS

FIG. 1A and FIG. 1B are views of the apparatuses for detecting luminescence from biological systems in response to magnetic fields according to the first embodiment of this invention.

FIG. 2 is a graph showing the result of measurement using the apparatus shown in FIG. 1A.

FIG. 3 is a view of the apparatus for detecting luminescence from biological systems in response to magnetic fields according to the second embodiment of this invention.

### DETAILED DESCRIPTION OF THE PREFERRED EMBODIMENT

In the following, with reference to the drawings, embodiments of this invention will be described. Same numerals in the drawings designate same corresponding parts.

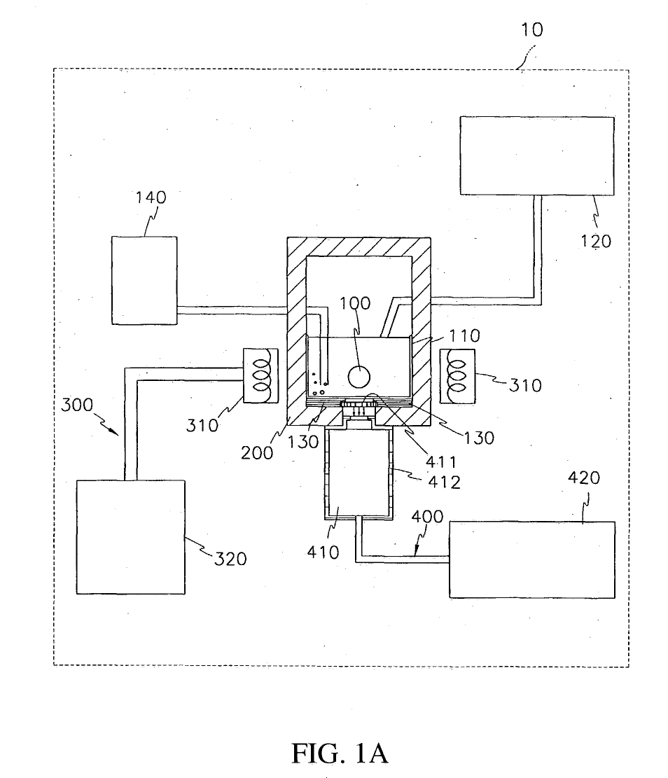

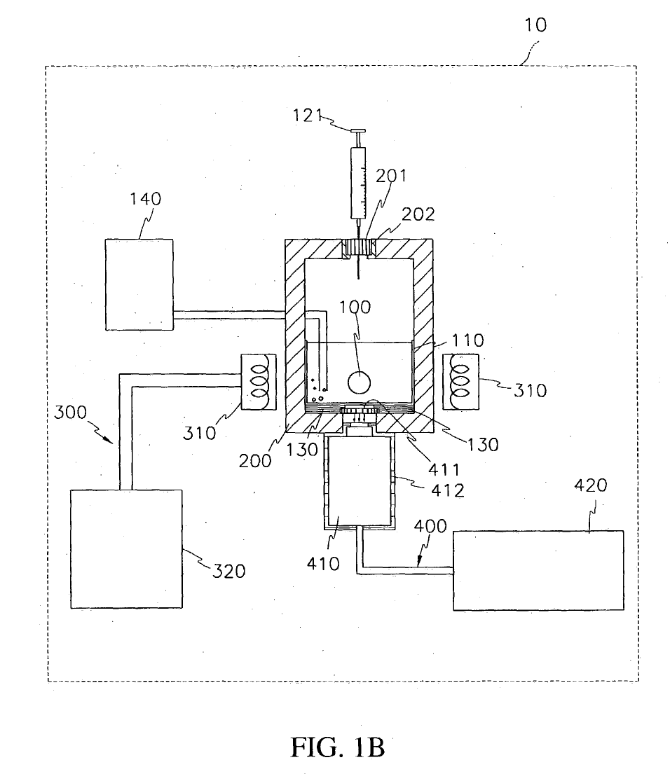

FIG. 1A is a view of an apparatus for detecting luminescence from biological systems in response to magnetic fields according to the first embodiment of this invention. The apparatus according to the first embodiment which is to measure biological samples such as biological tissues separated from living things comprises a magnetic field generator (300) which impresses a magnetic field on the biological sample (100), a photodetector (400) which detects luminescence emitted from the biological sample (100) when the magnetic field is impressed on it, and a dark box (200) which shields the biological sample (100) from external light.

In this embodiment, a biological sample (100) which is tissue separated from a living thing is put into a container (110) with a suitable buffer solution, and arranged in a dark box (200). If the said biological sample (100) is a biological tissue separated from a living thing, it is possible to provide luminous material from outside the dark box (200). In FIG. 1A, a luminous material provider (120) provides luminous material such as tBHP(tert-butylhydroperoxide), lucigenin, or luminol to the biological sample (100).

A constant-temperature unit (130) is provided to maintain the biological sample temperature similar to the body temperature of the living thing from which the biological tissue was separated to make the said biological tissue live longer. It maintains the container (110) at a constant temperature by circulating water of suitable temperature or by installing heating wires. The constant-temperature unit (130) may be installed on the bottom or the wall of the dark box (200) in which the biological sample (100) is placed, or may be installed in the container (110).

An oxygen and carbon dioxide provider (140) provides oxygen and carbon dioxide constantly to make the tissue cells of biological sample (100) live longer. The ratio of oxygen to carbon dioxide is preferably 95 to 5.

The magnetic field generator (300) comprises a signal generator (320) and a magnetic field generating coil (310). The signal generator (320) generates signals of which the frequency, amplitude, and shape such as sine wave, rectangular wave, triangular wave, and pulse wave can be controlled to impress the desired form of magnetic field. The magnetic field generating coil (310) receives these signals, and generates the magnetic field to impress on the biological sample (100) according to the signals.

The photodetector (400) which detects extremely small amount of light emitted from the biological sample (100) comprises a photomultiplier tube (410) and a data counting unit (420). The photomultiplier tube (410) multiplies the extremely small amount of light signal by the effect of secondary emission of electrons, then outputs corresponding electric pulse signals. The data counting unit (420) counts the pulse signals per unit time. The photomultiplier tube (410) is arranged underneath the container (110) which contains the biological sample (100) to prevent it from being influenced by the vapor from the buffer solution. The photomultiplier tube (410) may be prevented from being influenced by magnetic fields by wrapping in a shielding case (412) made of shielding material such as gunmetal. The time intervals of measurement may be controlled by attaching a shutter (411) before the photomultiplier tube (410). In this embodiment, although it is stated above that the photodetector (400) comprises a photomultiplier tube (410) and a data counting unit (420), it is not restricted to that. The photodetector (400) may comprise another suitable photodetecting device.

The apparatus shown in FIG. 1B is similar to that of FIG. 1A, but contains a part for using a syringe to provide luminous material manually instead of by a luminous material provider (120). This part is made by forming an opening for a holder (202) in the middle of top side of the dark box (200) and positioning a septum (201) made of rubber between the holder (202) and the dark box (200). The luminous material is provided by injecting a syringe (121), which is shaded, into the septum (201).

In the following, the operation of the apparatus as shown in FIGS. 1A and 1B for detecting luminescence from biological systems in response to magnetic fields will be described.

The biological sample (100) which is a tissue or cells separated from a living thing is crushed and dispersed in a suitable buffer solution, put in the container (110), and arranged in the dark box (200). It is then adapted to the dark surroundings for a specified time. To make the tissue cells of biological sample (100) live longer, the constant-temperature unit (130) maintains the temperature similar to the body temperature of the biological sample (100), and the oxygen and carbon dioxide provider (140) provides oxygen and carbon dioxide. Under these circumstances, the quantity of photons is measured by the photomultiplier tube (410) while impressing the magnetic field generated by the magnetic field generator (300) on the biological sample (100). At this time, the luminous material such as tBHP, lucigenin, or luminol may be provided to the biological sample (100) through the luminous material provider (120) or syringe (121) to increase the amount of light emitted from the biological sample (100).

If the magnetic field is impressed on the biological sample (100), the quantity of photons detected by the photomultiplier tube (410) becomes greater than that before the impression of the magnetic field. This happens through the following process. The biological tissue is stressed by the magnetic field, which causes the biological tissue to secrete toxic materials, and the toxic materials decompose the cells of the biological tissue. The emission of photons caused by the decomposition of the cells then increases the quantity of photons detected by the photomultiplier tube (410).

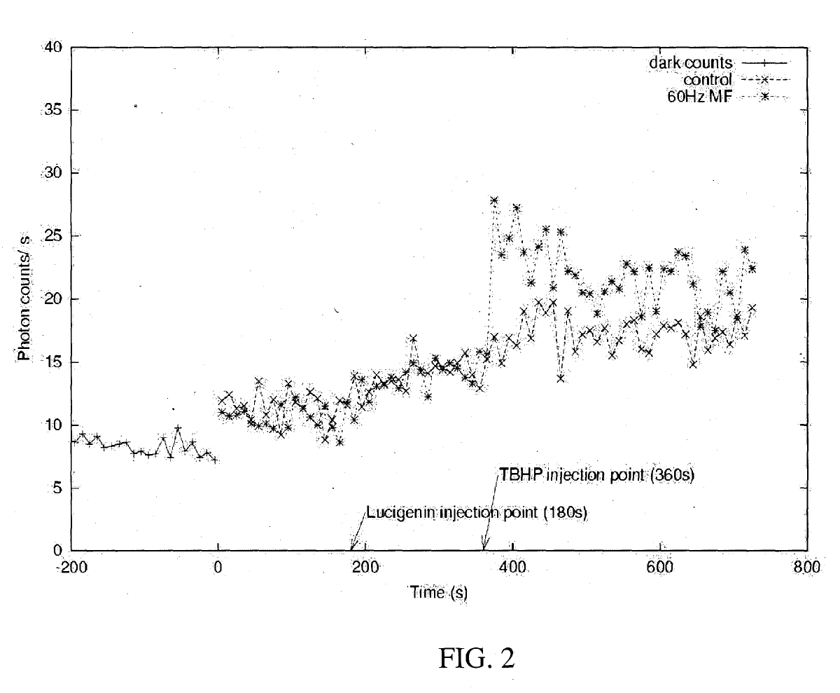

FIG. 2 is a graph which shows the result of measurement with mouse brain according to the first embodiment of this invention. The conditions of the measurement will be described in the following.

a. A mouse was sacrificed by decapitation, and the brain of the mouse extracted.

b. The tissue of the mouse brain was put into 20 ml of 0.05M tris-HCL buffer solution, and crushed and dispersed about 20 times with a homogenizer to become a brain homogenate.

c. 1.4 ml of the brain homogenate was put in a petri dish, placed in the dark box (200), and then adapted to the dark surroundings for 2 minutes.

d. The brain homogenate was maintained at the temperature of 37.5° C by the constant-temperature unit (130), and provided with oxygen and carbon dioxide with the ratio of 95 to 5 by the oxygen and carbon dioxide provider (140).

e. The quantity of photons was measured by the photomultiplier tube (410) in time intervals of 0.1 sec.

f. A 60 Hz sine wave magnetic field having 100 Gauss of flux density was impressed.

g. 180 seconds after the impression of the magnetic field, lucigenin was provided, and 360 seconds after the impression, 70% concentration of tBHP was provided.

In FIG. 2, the (+) mark line (before 0 s) shows dark counts before placing the brain homogenate in the dark box (200), the (x) mark line shows the photon counts without the impression of the magnetic field, and the (*) mark line shows the photon counts with the impression of the magnetic field. As shown in FIG. 2, the amount of light emitted from the brain homogenate with the impression of the magnetic field is larger than that without the impression of the magnetic field. From this result, the fact that the amount of light emitted from biological systems increases with the impression of magnetic fields on the biological systems is verified.

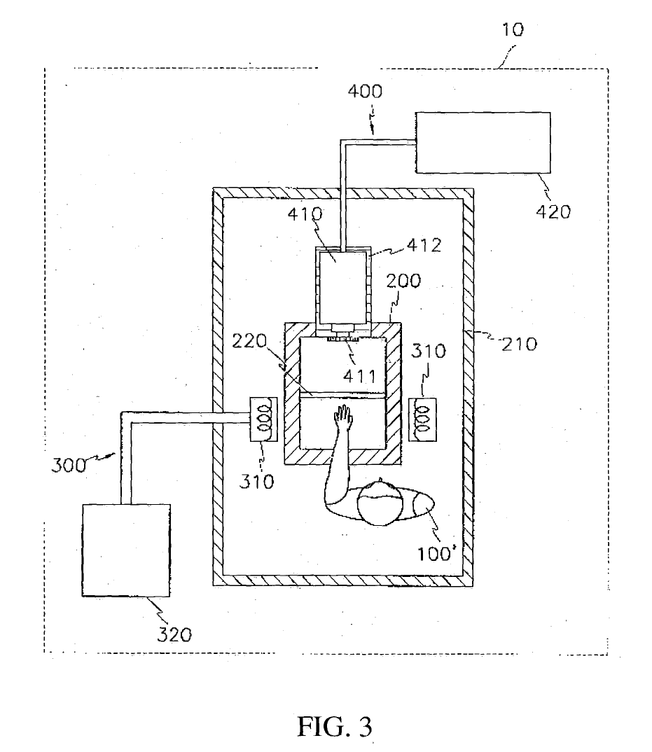

In the above stated embodiment, the subject of the measurement is a tissue or cells separated from a living thing. However the measurement can also be done without separation of tissue from a living thing. FIG. 3 is a view of the apparatus for detecting luminescence from biological systems in response to magnetic fields according to the second embodiment of the present invention. The apparatus in the second embodiment is devised to measure the effect of a magnetic field without separation of tissue from a living thing. The apparatus comprises a magnetic field generator (300) which impresses a magnetic field on a living thing (100′) or a part of a living thing, a photodetector (400) which detects luminescence emitted from the specific part of the living thing (100′) when the magnetic field is impressed, and a dark box (200) which shields the part to be measured from external light. The living thing (100′), the dark box (200), and the photomultiplier tube (410) are placed in a dark room (210). An infrared filter (220) may be placed between the part to be measured and the photodetector (400) to intercept the infrared light emitted from the living thing (100′). The magnetic field generator (300) and photodetector (400) are the same as those of the above first embodiment.

In the following, the operation of the apparatus according to the second embodiment will be described. The living thing (100′) has to be adapted to the dark room for a specified time—for example 10 minutes—to measure precisely the quantity of photons emitted from the living thing (100′) when impressed by the magnetic field. The part to be measured of the living thing (100′) adapted to the dark room is positioned in the dark box (200). The infrared filter (220) may be placed to eliminate noise caused by the infrared light. Under these circumstances, the measurement of the quantity of photons emitted from the living thing (100′) is performed through the photomultiplier tube (410) with the impression of the magnetic field generated by the magnetic field generator (300). The quantity of photons emitted with the impression of the magnetic field is greater than that without the impression of the magnetic field, and the effect of the magnetic field impressed on the living thing will be found by analyzing this result.

By using the apparatus for detecting luminescence from biological systems in response to magnetic fields, the response of cells, tissues, or living things to magnetic fields can be examined. For example, research has been undertaken about luminous characteristics of carcinoma cells in comparison with normal cells (ref. Motohiro Takeda and Humio Inaba, “A novel method of assessing carcinoma cell proliferation by biophoton emission”, Cancer Letters 127, 155-160, 1998). We may develop this research to investigate the remedy of cancers by comparing cancer cells on which magnetic fields are impressed with those on which magnetic fields are not impressed. Also, we can investigate the effects of magnetic fields on other diseases using similar methods.

This invention of the apparatus and method for detecting luminescence from biological systems in response to magnetic fields shows the relation between magnetic fields and biological oxidation stresses in real time, so if the increase of photons when a magnetic field is impressed is not significant, we can say that the stress caused by the magnetic field is relatively small.

Moreover, this invention can be applied to medical remedial and diagnostic equipment, because it can impress magnetic fields and check the response from living bodies.

In the above statements, we explained the technical features of the present invention with a few specific embodiments, but this invention is not limited to those embodiments. It is obvious that a person having ordinary skill in the art to which this invention pertains can modify or change this invention within the idea of this invention.

What is claimed is:

1. An apparatus for detecting luminescence from biological systems in response to magnetic fields comprising:
a magnetic field generator which is placed adjacent to a biological sample and generates a magnetic field to be impressed on said biological sample;
a photodetector which detects luminescence from said biological sample on which magnetic field is impressed by said magnetic field generator; and
a dark box which shields said biological sample from external light.

2. The apparatus for detecting luminescence from biological systems in response to magnetic fields as described in claim 1, wherein
said biological sample is a biological tissue or cells separated from a living thing.

3. The apparatus for detecting luminescence from biological systems in response to magnetic fields as described in claim 2, further comprising a constant-temperature unit which maintains said biological sample at an appointed temperature.

4. The apparatus for detecting luminescence from biological systems in response to magnetic fields as described in claim 2, further comprising a luminous material provider which provides luminous material to said biological sample.

5. The apparatus for detecting luminescence from biological systems in response to magnetic fields as described in claim 2, further comprising a syringe which provides luminous material to said biological sample.

6. The apparatus for detecting luminescence from biological systems in response to magnetic fields as described in claim 2, further comprising an oxygen and carbon dioxide provider which provides oxygen and carbon dioxide to said biological sample.

7. The apparatus for detecting luminescence from biological systems in response to magnetic fields as described in claim 1, wherein
said biological sample is a living thing.

8. The apparatus for detecting luminescence from biological systems in response to magnetic fields as described in claim 7, further comprising a infrared filter which is placed between the part to be measured and the said photodetector to intercept the infrared light emitted from said living thing.

9. The apparatus for detecting luminescence from biological systems in response to magnetic fields as described in claim 1, wherein
said magnetic field generator includes a signal generator and a magnetic field generating coil.

10. The apparatus for detecting luminescence from biological systems in response to magnetic fields as described in claim 1, wherein
said photodetector includes a photomultiplier tube and a data counting unit.

11. A method for detecting luminescence from biological systems in response to magnetic fields comprising the steps of:
(a) preparing a biological sample;
(b) shading said biological sample from external light;
(c) impressing a magnetic field on said biological sample; and
(d) detecting luminescence from said biological sample.

12. The method for detecting luminescence from biological systems in response to magnetic fields as described in claim 11, wherein
said biological sample is a biological tissue or cells separated from a living thing.

13. The method for detecting luminescence from biological systems in response to magnetic fields as described in claim 12, wherein said preparing step includes the steps of:
(a) maintaining said biological sample at a certain temperature;
(b) providing luminous material to said biological sample; and
(c) providing oxygen and carbon dioxide to said biological sample.

14. The method for detecting luminescence from biological systems in response to magnetic fields as described in claim 11, wherein
said biological sample is a living thing.
